(19) United States
(12) Patent Application Publication
(10) Pub. No.: US 2004/0248265 A1
(43) Pub. Date: Dec. 9, 2004

(54) Endophytes for production of podophyllotoxin

(76) Inventors: John R. Porter, Folsom, PA (US); Amy L. Eyberger, Newark, DE (US)

Correspondence Address: REED SMITH LLP, 2500 ONE LIBERTY PLACE, 1650 MARKET STREET, PHILADELPHIA, PA, 19103, US

(21) Appl. No.: 10/455,844

(22) Filed: Jun. 6, 2003

(51) Int. Cl. C12P 17/18; C12N 1/18

(52) U.S. Cl. 435/119; 435/254.2

(57) ABSTRACT

The present invention discloses podophyllotoxin producing endophytic fungi isolated from Podophyllum species. The invention provides methods for identifying podophyllotoxin producing endophytic fungi and methods for recovering podophyllotoxin from such fungi.

### FIELD OF THE INVENTION

The present invention relates to podophyllotoxin from natural sources. Specifically, the present invention relates to endophytic fungi capable of producing podophyllotoxin, methods of isolating such endophyte fungi and production of podophyllotoxin from the fungi.

### BACKGROUND OF THE INVENTION

Podophyllotoxin (PPT) is one of the most important plant products for the treatment of a variety of cancers. The compound is used as the precursor for the semi-synthesis of the anticancer compounds etoposide, teniposide, and etopophos (Schacter et al., 1994; Katz, 2002). These drugs, either singly or in combination with other anticancer agents, are in high demand for the treatment for a wide variety of cancers (Brooks & Alberts, 1996; Canel et al., 2000). Additional podophyllotoxin derivatives, such as NK611 (Damayanthi & Lown, 1998), are in human clinical trials for cancer chemotherapy, and there is a continuing search for additional derivatives with enhanced efficacy or reduced side-effects (e.g., Gordaliza et al., 2000; Arimondo et al., 2001; vanVliet et al., 2001).

However, there is a worldwide shortage of the parent compound, podophyllotoxin. The reasons for this are principally 1) because the compound has not been successfully synthesized on a commercial scale (Damayanthi & Lown, 1998; Berkowitz et al., 2000), 2) biotechnological approaches using tissue or cell culture of multiple species have been disappointing (Berlin et al., 1988; Chattopadhyay et al., 2002; Empt et al., 2000; Giri et al., 2001; Petersen & Alfermann, 2001), 3) agricultural production of the source plants have so far been equally unsuccessful (Maqbool et al., 2001; Moraes et al., 2001; Moraes et al., 2002), and 4) the collection of the source plants (P. hexandrum and P. peltatum) results in plant and population destruction because the underground rhizomes are the tissue with the highest content (Singh et al., 2001). Over-collection has already led to endangered species status for P. hexandrum (Bhadula et al., 1996; Maqbool et al., 2001; Singh et al., 2001), and populations of P. peltatum have been locally exterminated. Demand for podophyllotoxin is expected to rise because of its increasing use in synthesizing cancer drugs. A suitable alternative source would help alleviate the shortages in the production of this important compound.

Several endophyte fungi from yew trees capable of producing paclitaxel, an active component of Taxol®, have been isolated (Strobel et al., 1997; Strobel and Long, 1998; Strobel and Hess et al., 1996). Their potential as an effective alternative or novel source for therapeutic compounds has been recognized (Strobel and Long, 1998).

Similarly, the discovery of a podophyllotoxin-producing endophyte could lead to an alternative source of this anticancer drug precursor that is cheaper and unlimited.

### SUMMARY OF THE INVENTION

In the present invention, an alternative source of podophyllotoxin has been found. Specifically, the present invention discloses isolation of selected endophytes capable of producing podophyllotoxin. A total of 18 morphological isolates of endophyte fungi were isolated from mayapple root, rhizome, stem, and leaf tissues. The toxin was recovered from two of the extracts derived from the two different isolated fungi, thus far designated as Phialocephala fortinii strain PPAEE1 (ATCC as Accession No. PTA-5208) and PPAEE2 (ATCC as Accession No. PTA-5209).

Accordingly, in one aspect of the invention, a biologically pure fungal strain capable of producing podophyllotoxin is provided. The fungal strain occurs naturally as an endophytic fungus of a Podophyllum species such as P. peltatum. The preferred strains are those designated as PPAEE1 and PPAEE2 which have been deposited at ATCC as Accession Nos PTA-5208 and PTA-5209.

In another aspect of the invention a method for isolating an endophytic fungus capable of producing podophyllotoxin is provided. To isolate the podophyllotoxin producing endophytic fungus, an explant from a Podophyllum species is surface-sterilized and incubated on a growth medium under suitable conditions until mycelial growth of a fungus occurs. Then, a sample of fungal hyphae of the fungus is isolated and cultured in a nutrient medium and under culture conditions until a biomass of the fungus occurs. The fungal hyphae grow into mycelia in a matter of a few weeks, for example, in about 3 or 4 weeks. The mycelium or fungal biomass obtained after a few weeks of culture period is separated from the medium in which the hyphae grew for a few weeks. the fungal biomass and the medium are processed separately to obtain extracts of each. The extracts are then analyzed. For example, as part of the analysis, a chromatograph or mass spectrum of each of the extracts is obtained and compared with the chromatograph or mass spectrum of the podophyllotoxin standard. If the extract(s) show podophyllotoxin, then the endophytic fungus from which the extract was obtained is selected as the one capable of producing podophyllotoxin.

In yet another aspect of the invention, a method for the production of podophyllotoxin is provided. The method includes (a) culturing an endophyte fungus isolated from a Podophyllum species and capable of producing podophyllotoxin in a nutrient medium capable of supporting growth of the endophyte; and (b) concentrating and recovering the podophyllotoxin from the medium in which the endophyte has grown. The culture can be set up as a fermentation culture for large-scale production of the toxin.

### BRIEF DESCRIPTION OF THE DRAWINGS

FIG. 1 shows HPLC analysis of the Wild Rhizome SPE extract from an endophyte, designated PPAEE1, of Podophyllum peltatum. A. Analysis of the Wild Rhizome I SPE extract by the HPLC long method. B. Peak apex spectrum at 32.64 min from 190-400 nm observed in the analysis of the extract by the HPLC long method.

FIG. 2 shows an LC/MS analysis of the 50 μg/mL PPT standard and the Wild Rhizome SPE extract from an endophyte, designated PPAEE 1, of Podophyllum peltatum. A. Chromatograph of the initial HPLC separation of the 50 μg/mL PPT standard. B. Chromatograph of the initial HPLC separation of the Wild Rhizome extract.

FIG. 3 shows a Mass spectrum analysis of the peak at 20.9 min in Wild Rhizome SPE extract (FIG. 3A) and the 50 μg/mL PPT standard (FIG. 3B) peak at 20.95 min.

FIG. 4 is a flowchart describing the analysis of endophyte cultures for the presence of PPT.

### DETAILED DESCRIPTION OF THE INVENTION

The present invention discloses endophytic fungi obtained from Podophyllum species as a source of podophyllotoxin. An immediate advantage that can be envisioned from this finding is that these endophytic fungi, after isolation from their hosts, can be grown in fermentation cultures for large scale production of podophyllotoxin.

To this end, the present invention provides a method for isolating endophytic fungi which produce podophyllotoxin from Podophyllum species Podophyllum hexandrum, the Indian mayapple, and P. peltatum, the American mayapple or American mandrake. These plants can be collected from locations where the plant is known to grow naturally. For example, plants of P. peltatum can be gathered from relatively undisturbed wetland areas in northern Delaware. The information on locations that are known to naturally support growth of Podophyllum species can be obtained from standard taxonomy textbooks.

After collecting the Podophyllum species, the following standard procedures may be followed to isolate fungus and the podophyllotoxin from it: The plants are sectioned and sorted into rhizomes, roots, stems and leaves. These sections are treated or disinfected to remove any epiphytic organisms. Sections of the plant tissues are then cultured on standard growth media and under congenial growth conditions to isolate fungi. If fungal endophytes are found, these endophytes may be cultured for a few weeks before analyzing to determine if the endophytes were producing the secondary metabolite, PPT. Culture extracts are then tested for the presence of PPT using analytical tools such as HPLC. If podophyllotoxin is detected, the fungus is selected and grown in cultures, such as fermentation cultures, for large scale and sustained production of podophyllotoxin.

To grow a pure culture of an endophyte fungus, Podophyllum plant tissue sections or explants are incubated on agar media and maintained under standard growth conditions. For example, malt extract agar or Sabouraud's dextrose agar (SDA) medium containing sugars and a mixture of amino acids can be used as culture medium to grow an endophyte fungus of the present invention. A variety of standard fungal culture media are commercially available, for example, from sources such as Becton Dickinson & Co. (Franklin Lakes, N.J.).

The following are some of the typical microbial culture media that can be used to culture the endophyte fungi of the present invention.

A. Malt Extract Agar

(Formula per liter)

Maltose 12.75 g

Bacto Dextrin 2.75 g

Bacto Glycerol 2.35 g

Bacto Peptone 0.78 g

Bacto Agar 15 g

Final pH 4.7±0.2 at 25° C.

B. Malt Extract Broth

(Formula per liter)

Malt Extract Base 6 g

Maltose 1.8 g

Bacto Dextrose 6 g

Bacto Yeast Extract 1.2 g

Final pH 4.7±0.2 at 25° C.

C. Potato Dextrose Agar

(Formula per liter)

Potatoes, Infusion from 200 g

Bacto Dextrose 20 g

Bacto Agar 15 g

Final pH 5.6±0.2 at 25° C.

D. Sabouraud Dextrose Agar

(Formula per liter)

Bacto Neopeptone 10 g

Bacto Dextrose 40 g

Bacto Agar 15 g

Final pH 5.6±0.2 at 25° C.

E. YM Agar (YMA)(=Yeast Extract Malt Extract Agar)

(Formula per liter)

Bacto Yeast Extract 3 g

Bacto Malt Extract 3 g

Bacto Peptone 5 g

Bacto Dextrose 1 g

Bacto Agar 20 g

Final pH 6.2±25° C.

F. YM Broth (YMB)(=Yeast Extract Malt Extract Broth)

(Formula per liter)

Bacto Yeast Extract 3 g

Bacto Malt Extract 3 g

Bacto Peptone 5 g

Bacto Dextrose 10 g

Final pH 6.2±0.2 at 25° C.

The incubation of the fungus can be carried out, for example, at 23-25° C., in the dark. More than one type of fungus may grow in each culture. Different types of endophytic fungi may be identified based on their morphological differences, such as color of the fungal colony, growth rate, and colony surface (sparse, disperse, matted, aerial, submerged). Typically, fungi of the invention in culture are dark brown to black in color, although some are green, tan or light brown, pink, white to light grey, yellow, and orange. In such cases, the fungal mycelia of each type are isolated and subcultured to raise a pure culture.

For example, the fungal mycelia of Phialocephala fortinii strains PPAEE1 PPAEE2 can be readily identified by their morphological features. An examination of the morphological features of the fungus designated as P. fortinii strain PPAEE 1 indicates that it grows as arborescent mycelia on malt extract agar with aerial, surficial and submerged hyphae. The principal mycelium color is dark olive green to black with hyaline growing tips. The aerial and surfacial hyphae are slender with pointed tips and are either black or medium tan in color. These hyphae are septate, with cells 1.5-3.5 μm wide and 20-35 μm long. The submerged hyphae is septate and composed of subtoroid to toroid cells, 3.5-4 pm wide and 8 μm long; the principal color of these hyphae is dark brown. The sporulation is sparse, even after more than one year in culture at 4° C. The conidia are hyaline, spherical, and 1.5-3.5 μm in diameter.

An examination of the morphological features of the fungus designated as P. fortinii strain PPAEE2 indicates that it grows as a more typical tufted mycelium on malt extract agar. There are numerous aerial, surficial and submerged hyphae all of which are highly and irregularly branched. The mycelia are dark brown to black with dark tan, cottony centers. Almost all of the aerial hyphae and some of the surficial hyphae are medium to dark tan, septate, with cells 4-6 μtm wide by 25-30 μm long, and with slender, pointed, hyaline tips. The surface hyphae and some of the submerged hyphae are hyaline, tan, or brown to black, and consist of cells that are shorter and broader (6-8 μm wide by 16-18 μm long), that are somewhat swollen in the center (subtoroid), and with slender, pointed, hyaline growing tips. The abundant submerged hyphae are hyaline to dark brown or black, slender, and with pointed growing tips. Many of the submerged hyphae consist of cells 4-5 μm wide by 8-10 μm long that are slightly wedge shaped over their length and are distinctly articulated at the septac. There is moderate sporulation after one year in culture at 4° C. The conidiophores are subglobose, dense, 17-18 μm wide by 20 μm long. The conidia are spherical, 2-3 μm in diameter. Some cells on the surficial hyphae have terminal cells with what appear to be internal oil droplets along their length. These droplets are approximately the size of the conidia, but some are slightly larger and some are much smaller. These cells give an overall impression of asci, but are not convincing as these structures.

For purposes of clarity, the term “hypha” (pl. hyphae) refers to long, slender, branched or unbranched filaments that make up the body of a fungal colony. The term “mycelium” (pl. mycelia) refers to the collected hyphae of a single fungal colony. The term “biomass” refers to the amount, weight or volume of the product of the growth of a fungus.

Once a pure culture of an endophyte is obtained, the culture is grown in a suitable medium to grow a fungal biomass. For example, a fungal mycelium is inoculated into one liter of YMB medium in large surface volume flasks. One flask of each fungus is placed in a growth room without agitation (still culture) or placed on a rotating platform shaker at 100 rpm. The shaking serves to increase the aeration of the culture. Aerated culture conditions are more preferred than still cultures.

At the end of the culture period, for example 4-6 weeks, the fungal biomass can be harvested by centrifugation or filtration, as appropriate. Fungi that grow on the surface of the medium can also be collected by vacuum filtration. In such instances, some clean-up of the culture medium by centrifugation may still be required. However, fungi that grow as diffuse mycelia throughout the medium volume are best collected in a centrifugation step. Centrifugation can be conducted in polycarbonate bottles (Sorvall RC5B, GSA rotor). The filtered biomass or the centrifugation pellet may then be transferred to blotter paper, allowed to blot briefly (e.g., <5 min) and the biomass weighed to determine biomass fresh weight. This fresh weight may also be used as the starting point in the optimization of growth and production. The fungal biomass is then dried in an oven overnight for determination of dry weight. One skilled in the art would know how to determine dry weight and the percent dry weight. The dry biomass is reduced to powder by grinding. The filtered medium is reduced to dry medium solids by freezing the medium and removing the water by lyophilization (freeze-drying) (e.g., by Labconco Lyph-Lock 12).

Generally speaking, an “extract” is a product of interest obtained after discarding certain portions during an extraction procedure. An “extraction” can be synonymous with extract but more generally refers to a stepwise procedure or protocol by which one or more aqueous or non-aqueous solvents are used to sequentially separate and isolate the chemical constituents of a sample. Differential solubility of different compounds of the original sample in the various solvents leads to separation of the various constituents into separate fractions. The extraction protocol can be confined to liquid solvents with stirring, shaking or trituration (rapid, vigorous mixing), as well as procedures which use a special apparatus (e.g., Soxhlet), a chromatographic step (column chromatography, flash chromatography, solid phase extraction (SPE)), or a step in which the pH of the solution is modified in a specific way. Discarded portions during an extraction procedure may include the insoluble biomass (containing compounds of a structural nature or which are not soluble in the solvents chosen), compounds which are insoluble at a particular step, or extracts which are deemed by the operator not to be of value for analysis.

In the context of the present invention, the biomass powder and the medium solids can be extracted, for example, with ethanol (e.g., 95%) by Soxhlet extraction to obtain an extract containing the compound of interest, i.e., podophyllotoxin. It is preferred, however, that the biomass powder be processed by Soxhlet extraction and spent culture media or medium solids be processed by solid phase extraction (SPE).

Soxhlet extraction is a procedure for isolating and concentrating thermostable compounds from finely divided tissue through sequential flushing with freshly distilled solvent. The procedure uses a specialized glass apparatus known as the Soxhlet apparatus and it is well known in the art. The time of extraction can vary, but generally continues for about 8-36 hours. The biomass is ground or powdered to increase the surface area for contact with the solvent. The extracted materials collect in the solvent flask until the end of extraction. Following extraction, the extract is cooled and may be analyzed as is, reduced to a smaller volume to concentrate the compounds before analysis, or further processed for additional isolation and separation of the desired compounds. It is preferred that the extract is filtered, brought to a standard volume, and then subjected to HPLC analysis with authentic podophyllotoxin (Sigma) as an external standard.

SPE (solid phase extraction) is a liquid chromatographic method for concentrating and purifying a sample component and it is well known to one skilled in the art. Briefly, for example, the SPE column or cartridge consists of a small column or barrel (often a plastic syringe barrel) containing a measured amount of a chromatographic medium, such as octadecyl (C18)-modified silica. The amount of solid medium is often 100-500 mg. A frit at the barrel outlet prevents the medium from being lost during the extraction. A second frit at the top of the medium bed prevents disturbing the sample bed on loading the sample. The bed is charged by flushing with solvent and/or water, depending on the nature of the medium and the sample. The sample, containing the compound(s) of interest, is loaded in as small a volume as reasonable onto the column bed. The column is then flushed with a relatively large volume (5-20 mL) of a weak solvent that will not elute the compound(s) of interest (often HPLC grade water). After the wash step, compounds with less attraction to the bed medium are eluted with one or more steps of a stronger solvent up to a point at which none of the compound of interest will be lost. For example, podophyllotoxin begins eluting at 40% ethanol. The bed is washed with up to 35% ethanol to get rid of more polar contaminants but retain the podophyllotoxin. The compounds of interest are then eluted with a small volume (0.1-1.0 mL) of a solvent which is strong enough to elute all of the materials of interest (along with some, but fewer, contaminants) (e.g.,40-50% ethanol for podophyllotoxin). The column bed may then be regenerated by washing with strong solvent, such as 100% ethanol and then washed with the initial solvent (HPLC grade water) to return to the starting conditions. This is generally a clean-up step, so the washes before and after the sample elution step are discarded and only the sample, in a small volume of the elution solvent, is retained. The conditions for running an SPE clean-up are determined with known amounts of authentic podophyllotoxin prior to using the method on a sample with an unknown quantity of podophyllotoxin. Initial, scouting HPLC runs can help confirm that the podophyllotoxin amount in the sample, if any, is such that it will not exceed the capacity of the SPE column.

For the extraction of podophyllotoxin from dry biomass and/or medium solids, solvent extraction method is preferred. Of the solvents, ethanol is preferred, particularly 95% ethanol or ethanol with a polarity (solvent strength) index (P′) of about 4.6 is preferred. Other suitable solvents would include ethyl acetate, preferably 97% ethyl acetate, tetrahydrofuran, preferably 90% tetrahydrofuran, n-propanol, preferably 90% n-propanol, 2-propanol, preferably 89% 2-propanol, methylene chloride:methanol, preferably 25:75 methylene chloride:methanol, or ethyl acetate:acetonitrile, preferably 85:15 ethyl acetate:acetonitrile. Suitable solvents for the elution of podophyllotoxin from a SPE column should have a polarity approximating a range of 40-60% ethanol in water, or a range of P′ of 6.6-7.8. This would include such solvents as 2-propanol, preferably 38-57% 2-propanol; n-propanol, preferably 38-58% n-propanol; methanol or acetonitrile, preferably 47-70% methanol or acetonitrile; dimethyl sulfoxide, preferably 80-100% dimethyl sulfoxide. When ethanol is used as a solvent, not only that podophyllotoxin begins eluting at 40% ethanol, as described above, the majority of podophyllotoxin will elute with 40% ethanol. The use of 50-60% ethanol removes any residual amounts. In a preferred embodiment of the invention, column washing and elution steps may proceed with 30-35% wash and 50% elution, or the 40 and 50% elutions which can be pooled together. Hexane can also be used to wash the column because podophyllotoxin is not considered soluble in hexane.

While the solvent extraction method is prefferred, other extraction and separation/purification methods known in the art can be used in order to practice the present invention. For example, supercriticalfluid extraction with CO2, LH-20 chromatography, affinity chromatography or other extraction and separation/purification methods suitable for industrial scale production can be used.

The quantity of podophyllotoxin in the extract is determined by use of a calibration curve developed with authentic podophyllotoxin. The podophyllotoxin content of the biomass and the medium solids are then scaled to the original fresh weight, dry weight, and volume. This data may allow a precise determination of the level of podophyllotoxin production in the starting conditions for the fungi.

Both isocratic and gradient elution methods have been used in HPLC analysis of podophyllotoxin (Yoo & Porter, 1993, J Nat Prod 56(5):715-721; Canel et al., 2001, Phytochemistry, 54(2):115-120). Two different gradient methods, designated as the short and long methods may be used. The short method is used as a scouting tool, to preliminarily assess an extract for podophyllotoxin presence. When the sample is too complex, leading to overlapping peaks in the region where podophyllotoxin elutes, when the podophyllotoxin concentration is particularly low, or when there is a need to do LC-MS analysis, the long method may be used.

The short method, for example, may consist of analysis of 10-20 μL of extract in an acetonitrile gradient (20-80%) over 15 minutes (1 mL/min). Both the aqueous and organic phases are buffered with 0.1% trifluoroacetic acid (TFA). The column is 4.6 mm×250 mm with a C18 chemistry (5 μm particle size) and a C18 guard column; we have found that C18 columns from a variety of manufacturers are suitable for the analysis. Detection is done with a diode array detector (DAD), which allows recording multiple wavelengths of the peak to obtain an absorption spectrum. The detector software collects absorbance over the range of 190-400 nm. Monitoring of four spectra allows real time assessment of sample complexity. Determination of the presence of podophyllotoxin is through elution at a time and a UV absorption spectrum corresponding to the elution time and UV spectrum of authentic podophyllotoxin.

The long method, for example, may consist of a 40 min linear gradient of 5-95% acetonitrile in water buffered with TFA (1 mL/min). The sample size, column, and detection remains the same as for the short method. Podophyllotoxin has generally been base-line resolved, well separated from interfering peaks, and gives clearer absorption spectra in the long method compared to the short method. Authentic podophyllotoxin samples are analyzed at the beginning of each day of analysis and quantitation curves are developed by varying the concentration of podophyllotoxin in the sample while keeping the injection volume constant. Thus, to determine the presence of PPT in samples analyzed by the HPLC long method, peak retention times can be compared to a given PPT standard. Samples containing peaks with retention times, for example, from 31-39 min may confirm the presence of PPT. It should be noted, however, that the absolute retention time is controlled by several variables, including the specific column, the system on which the sample is run, the solvent flow rate, temperature, solvent pH, and the like. For example, if a given sample is run on two different systems and three different columns, the retention rates may vary. Notwithstanding, one can always confirm putative identity of a peak based on comparison to authentic podophyllotoxin and at least the absorption spectrum. Of course, the absolute confirmation of podophyllotoxin can be conducted, for example, by comparison of retention times, absorption spectrum, and MS fragmentation pattern to authentic podophyllotoxin.

Besides the above analysis, activity of the isolated podophyllotoxin from the endophyte fungi of the invention may also be tested by carrying out suitable bioassay. Brine Shrimp Lethality (BSL) assay is one such bioassay known in the art. The BSL bioassay is a general cytotoxicity test that serves as an indication of potential anticancer activity of podophyllotoxin. The details on BSL bioassay can be found in the publication by Meyer et al., 1982, Planta Medica 45:31-34. Briefly, brine shrimp (Artemia salina) eggs are hatched in a commercial brine solution (InstantOcean or the like), under lights and with aeration, and with a small amount of yeast extract added as food. At 24 h from starting the culture, the brine shrimp nauplii (young shrimps) are collected by pipette. Generally 10 nauplli are collected at one time. These are transferred to a well of a 12-well or 24-well plate. The volume of the well is adjusted to a standard volume. A small amount of a test solution is added, at various concentrations (generally 0-100 μg/mL) to different wells. Each well receives the same volume of solution. The solvent in which the test samples are dissolved is added in a separate well (negative solvent control). A compound with known activity is also included at one or more concentrations to give a positive control. At least one well with no addition of solvent, test solution or positive control solution is used to look for background death rates (second negative control). After 24 h incubation at room temperature, the number of dead nauplii are recorded for each well. The percent kill is calculated based on the number of surviving nauplii (with correction for background death rates) and an EC₅₀ value is calculated (concentration that is effective in killing 50% of the population).

### EXAMPLES

The following examples further illustrate the present invention, but of course should not be construed as in any way limiting its scope. In other words, the examples are illustrative, but do not limit the invention. The examples below are carried out using standard techniques and procedures that are well known and routine to those of skill in the art.

### Example 1

### Podophyllotoxin from Endophytes of Podophyllum

In order to demonstrate the podophyllotoxin is produced de novo by the endophytic fungi found in Podophyllum species, the following procedures were carried out:

Preparation of Explants of Podophyllum

Two specimens of P. peltatum were obtained from sites in northern Delaware in early May of 1999. Plants, designated “wild” and “cultivated” were gathered from the state of Delaware. When harvested, both plants were mature and in bloom. To avoid overgrowth of epiphytic organisms and to minimize contamination of endophytic cultures, plants were transported to the laboratory within two hours after harvesting. Plants were then immediately prepared for the isolation of endophytes.

Whole plants were rinsed with deionized (DI) water and washed with a 1% solution of LiquiNox. Plants were divided into four groups of explants: leaf, stem, root and rhizome. Each group was then subjected to a three-step sterilization procedure (Petrini, 1986). Each group was placed in a 95% EtOH solution for 1 min, a 20% bleach solution for 3 min, fresh 95% EtOH solution for 0.5 min and a final 1 min rinse in sterile, filtered DI water. Using sterile forceps, each group was then placed in a sterile, metal container in a HEPA filtered laminar flow hood for sectioning. Approximately 1 cm long sections of root, rhizome and stem and 1 cm square sections of leaf were prepared with a sterile scalpel. Samples of each group, which had been washed but not surface sterilized, were also sectioned. This group served as a control to distinguish epiphytic organisms from true endophytes.

Incubation of Explants

Sections, also referred to herein as explants, of each plant group were placed on two different agar preparations and incubated under three different conditions. Agar preparations consisted of 1% malt extract agar (Difco #0112-17-6) and 1% malt extract agar with 0.1% streptomycin sulfate (Fisher Biotech #BP910-50). Streptomycin sulfate was added to prevent bacterial growth that could inhibit fungal growth. Plates were incubated at 20-23° C. in the dark, 20-23° C. in the light (wide spectrum fluorescent lamps GE F15T8/PL/AQ/WS) and 4° C. in the dark. These different incubation conditions were used in the hope that they would serve as an aid in classification of the endophytes isolated. A total of 169 explant cultures were prepared from the wild plant and 130 prepared from the cultivated plant (Tables 1 and 2). Cultures incubated at 20-23° C. in the dark and 20-23° C. in the light were monitored periodically for four to six weeks for the presence of fungal or bacterial growth. If fungal growth was observed, isolates were subcultured to fresh agar plates to obtain axenic cultures. If no growth was observed, cultures were discarded. Cultures incubated at 4° C. in the dark were maintained at this condition for one year to induce the sporulation of any sterile cultures (Petrini, 1986). Agar plates containing no plant sections were also incubated in each condition to serve as negative controls.

TABLE 1
Total Number of Wild Explant Cultures

| | Incubation Conditions | | |
|---|---|---|---|
| | 20° C.-23° C. | 4° C.-8° C. | 20° C.-23° C. |
| Wild Explant Cultures | Dark | Dark | Light |
| Root | | | |
| Surface Wash only/Malt | 2 | 2 | 2 |
| Agar | | | |
| Surface Sterilized/Malt | 10 | 3 | 3 |
| Agar | | | |
| Surface Wash only/Malt | 3 | 1 | 1 |
| Agar w/Streptomycin | | | |
| Surface Sterilized/Malt | 6 | 6 | 6 |
| Agar w/Streptomycin | | | |
| Rhizome | | | |
| Surface Wash only/Malt | 1 | 1 | 1 |
| Agar | | | |
| Surface Sterilized/Malt | 10 | 3 | 3 |
| Agar | | | |
| Surface Wash only/Malt | 1 | 1 | 1 |
| Agar w/Streptomycin | | | |
| Surface Sterilized/Malt | 6 | 3 | 3 |
| Agar w/Streptomycin | | | |
| Stem | | | |
| Surface Wash only/Malt | 3 | 2 | 2 |
| Agar | | | |
| Surface Sterilized/Malt | 10 | 3 | 3 |
| Agar | | | |
| Surface Wash only/Malt | 3 | 2 | 2 |
| Agar w/Streptomycin | | | |
| Surface Sterilized/Malt | 5 | 5 | 5 |
| Agar w/Streptomycin | | | |
| Leaf | | | |
| Surface Wash only/Malt | 3 | 2 | 2 |
| Agar | | | |
| Surface Sterilized/Malt | 10 | 3 | 3 |
| Agar | | | |
| Surface Wash only/Malt | 3 | 2 | 2 |
| Agar w/Streptomycin | | | |
| Surface Sterilized/Malt | 5 | 5 | 5 |
| Agar w/Streptomycin | | | |

TABLE 2
Total Number of Cultivated Explant Cultures

| | Incubation Conditions | | |
|---|---|---|---|
| Cultivated | 20° C.-23° C. | 4° C.-8° C. | 20° C.-23° C. |
| Explant Cultures | Dark | Dark | Light |
| Root | | | |
| Surface Wash only/Malt | 2 | 2 | 2 |
| Agar | | | |
| Surface Sterilized/Malt | 3 | 3 | 3 |
| Agar | | | |
| Surface Wash only/Malt | 1 | 1 | 1 |
| Agar w/Streptomycin | | | |
| Surface Sterilized/Malt | 4 | 6 | 6 |
| Agar w/Streptomycin | | | |
| Rhizome | | | |
| Surface Wash only/Malt | 1 | 1 | 1 |
| Agar | | | |
| Surface Sterilized/Malt | 3 | 3 | 3 |
| Agar | | | |
| Surface Wash only/Malt | 1 | 1 | 1 |
| Agar w/Streptomycin | | | |
| Surface Sterilized/Malt | 3 | 3 | 3 |
| Agar w/Streptomycin | | | |
| Stem | | | |
| Surface Wash only/Malt | 2 | 2 | 2 |
| Agar | | | |
| Surface Sterilized/Malt | 3 | 3 | 3 |
| Agar | | | |
| Surface Wash only/Malt | 2 | 2 | 2 |
| Agar w/Streptomycin | | | |
| Surface Sterilized/Malt | 5 | 5 | 5 |
| Agar w/Streptomycin | | | |
| Leaf | | | |
| Surface Wash only/Malt | 2 | 2 | 2 |
| Agar | | | |
| Surface Sterilized/Malt | 3 | 3 | 3 |
| Agar | | | |
| Surface Wash only/Malt | 2 | 2 | 2 |
| Agar w/Streptomycin | | | |
| Surface Sterilized/Malt | 5 | 5 | 5 |
| Agar w/Streptomycin | | | |

Fungal Culture

Isolates from each culture were stored by transferring agar plugs to a 15% glycerol solution and placed at −70° C. Primary cultures containing morphologically distinct fungal growth were chosen for further analysis. Isolates of primary cultures were mainly used for further analysis. However, if growth was too diverse to obtain a clean sample, secondary cultures were used. Secondary cultures were individual subcultures of explant cultures that contained multiple organisms. Secondary cultures were prepared to obtain better separation of isolates. Fungal isolates were then cultured in broth culture by transferring two or three, 0.5×0.5 cm agar plugs containing mycelia to 500 mL of malt extract broth (Difco #0113-17-5). These cultures were incubated at 20-23° C. in the dark under still conditions for a minimum of 4 weeks to ensure the accumulation of adequate biomass.

After 4-6 weeks of incubation in dark conditions at 20-23° C., explant cultures were analyzed for the presence of potential PPT-producing fungal endophytes. The total numbers of cultures from each plant section are listed in Tables 3 and 4. The isolates thought to be of bacterial origin were not further analyzed. Isolates observed on both surface-washed and surface-sterilized cultures were determined to be epiphytes and also not further analyzed.

Isolates remaining were cataloged and stored at 4° C. until further analysis as potential PPT-producing fungal endophytes. The number of fungal endophytes from wild explant cultures incubated at 20-23° C. that were analyzed for the ability to produce PPT was 13. Two of them were from root explants, 10 were from rhizome explants and one was from a leaf explant. No fungal endophytes were isolated from stem explants in this group. The total number of fungal endophytes from cultivated explant cultures incubated at 20-23° C. was five. One was from a root explant, three were from rhizome explants and one was from a leaf explant; no fungal endophytes were isolated from stem explants in this group. The endophytes isolated from the wild and cultivated explant cultures incubated in light conditions at 20-23° C. and in dark conditions at 4° C. were found not to be morphologically distinct from those isolated from the 20-23° C. dark condition. Therefore, no fungi from these conditions were further analyzed.

TABLE 3
Potential Endophytes of Wild Explants Incubated in Dark
Conditions at 20-23° C.

| | Total | Potential PPT- |
|---|---|---|
| | Number of | producing |
| Incubation Conditions | Cultures¹ | Endophytes² |
| Root | | |
| Surface Wash only/Malt Agar | 2 | 0 |
| Surface Sterilized/Malt Agar | 10 | 0 |
| Surface Wash only/Malt Agar | 3 | 0 |
| w/Streptomycin | | |
| Surface Sterilized/Malt Agar | 6 | 2 |
| w/Streptomycin | | |
| Rhizome | | |
| Surface Wash only/Malt Agar | 1 | 0 |
| Surface Sterilized/Malt Agar | 10 | 6 |
| Surface Wash only/Malt Agar | 1 | 0 |
| w/Streptomycin | | |
| Surface Sterilized/Malt Agar | 6 | 4 |
| w/Streptomycin | | |
| Stem | | |
| Surface Wash only/Malt Agar | 3 | 0 |
| Surface Sterilized/Malt Agar | 10 | 0 |
| Surface Wash only/Malt Agar | 3 | 0 |
| w/Streptomycin | | |
| Surface Sterilized/Malt Agar | 5 | 0 |
| w/Streptomycin | | |
| Leaf | | |
| Surface Wash only/Malt Agar | 3 | 0 |
| Surface Sterilized/Malt Agar | 10 | 0 |
| Surface Wash only/Malt Agar | 3 | 0 |
| w/Streptomycin | | |
| Surface Sterilized/Malt Agar | 5 | 1 |
| w/Streptomycin | | |

¹Total number of cultures containing microbial growth
²Cultures containing fungal growth considered to be potential PPT-producing endophytes.

TABLE 4
Potential Endophytes of Cultivated Explants Incubated in Dark
Conditions at 20-23°

| | Total | Potential PPT- |
|---|---|---|
| | Number of | producing |
| Incubation Conditions | Cultures¹ | Endophytes² |
| Root | | |
| Surface Wash only/Malt Agar | 2 | 0 |
| Surface Sterilized/Malt Agar | 3 | 0 |
| Surface Wash only/Malt Agar | 1 | 0 |
| w/Streptomycin | | |
| Surface Sterilized/Malt Agar | 4 | 1 |
| w/Streptomycin | | |
| Rhizome | | |
| Surface Wash only/Malt Agar | 1 | 0 |
| Surface Sterilized/Malt Agar | 3 | 2 |
| Surface Wash only/Malt Agar | 1 | 0 |
| w/Streptomycin | | |
| Surface Sterilized/Malt Agar | 3 | 1 |
| w/Streptomycin | | |
| Stem | | |
| Surface Wash only/Malt Agar | 2 | 0 |
| Surface Sterilized/Malt Agar | 3 | 0 |
| Surface Wash only/Malt Agar | 2 | 0 |
| w/Streptomycin | | |
| Surface Sterilized/Malt Agar | 5 | 0 |
| w/Streptomycin | | |
| Leaf | | |
| Surface Wash only/Malt Agar | 2 | 0 |
| Surface Sterilized/Malt Agar | 3 | 0 |
| Surface Wash only/Malt Agar | 2 | 0 |
| w/Streptomycin | | |
| Surface Sterilized/Malt Agar | 5 | 1 |
| w/Streptomycin | | |

¹Total number of cultures containing microbial growth
²Cultures containing fungal growth considered to be potential PPT-producing endophytes.

Separation of Fungal Biomass from the Liquid Culture Medium

To determine if an isolate was producing PPT, the fungal biomass was first separated from the culture medium by filtering through a 0.2 μm filter. Many isolates contained a biomass that was very fibrous and could not be filtered easily through a 0.2 μm filter. For these samples, the culture was divided into 125-250 mL aliquots and centrifuged at 8000 rpm for 6-8 hr (Sorvall RC-5B refrigerated Superspeed centrifuge with Super-Lite™ GSA rotor model #SAK-1500) before the culture supernatant were filtered. Remaining biomass was dried at 70-80° C. for 2-4 hr.

Processing to Obtain Extracts from Fungal Biomass by Soxhlet

The biomass was weighed and stored at 4° C. until it was processed. The biomass was crushed with a metal spatula to disrupt cells and aid the extraction process. The crushed biomass was extracted by Soxhlet in 95% ethanol for 6-8 hr. The extract was stored at 4° C. until analysis by HPLC.

Processing to Obtain Extracts from Culture Media by SPE

To prepare extracts from the spent culture media, solid phase extraction (SPE) was performed using an octadecyl (C₁₈) column (BakerBond #7020-13, 500 mg sorbent 6 mL volume wide mouth). Before the extraction, each column was conditioned with 5 mL of DI water, 5 mL of 100% EtOH and 5 mL DI water, successively. Ensuring the column did not become dry at any time, a filtered culture medium sample was then passed through the column. Depending on the characteristics of the culture media and ease of extraction, 250-500 mL of culture medium was passed through the column. If column flow became restricted, two columns were used and the extracts from both were pooled. After the culture medium was passed through the column, 5 mL of hexane was passed through the column to remove the more nonpolar compounds. Secondly, 5 mL of 30% EtOH was passed through the column. This was followed by 5 mL each of 40% EtOH and 50% EtOH. All fractions were collected and analyzed separately.

Optimization of the SPE procedure used to extract PPT from spent culture media was developed from experiments using PPT in aqueous solution. It was determined that the optimal concentration of EtOH needed to extract PPT from the SPE column was 40%. A 6 μg/mL solution of PPT in DI water was solid phase extracted as described elsewhere in the description of the present invention. The PPT sample was extracted using solutions of EtOH ranging from 10% to 100% in 5 mL aliquots and collected. All fractions were then analyzed by the initial screening HPLC method described above using a 10 μL injection volume and an isocratic mobile phase of 50% acetonitrile (HPLC grade) and 50% DI water. The gradient run and larger injection volume described above were modified for optimization experiments. An isocratic mobile phase and smaller injection volume was used due to the lower complexity and higher concentration of the PPT standard relative to extract samples. The majority of PPT was present in the 40% EtOH fraction.

Analysis of Soxhlet and SPE Extracts by Initial HPLC Screening Method

Soxhlet and SPE samples from 18 endophytes isolated from the wild and cultivated explant cultures were analyzed for the presence of PPT by the initial HPLC screening method. Samples were analyzed on one of two Hewlett Packard 1090M HPLC instruments (S/N 2650A01242 & S/N 2750A01903). Separation of extracts was carried out initially on a 4.6 mm i.d.×250 mm, 5 μm Zorbax ODS column (P/N 880952.702 S/N F46663) with a guard column. When separations could no longer be carried out on this column due to column failure, experiments were carried out using a replacement 4.6 mm i.d.×250 mm, 5 μm Zorbax SB-C18 (P/N 880975-902 S/N USCL008840) with a guard column. Spectrophotometric detection of PPT was at 254 nm using a diode array detector. Analysis of 20 μL of each extract was performed using a 15 min linear gradient of 20-80% acetonitrile (HPLC grade) and DI water mobile phase. The flow rate was 1 mL/min. At the beginning of each day that samples were to be analyzed by HPLC, a blank run (no injection) and a standard run of known PPT concentration (Sigma #P4405) were performed. These runs were performed to ensure column performance and that no residual sample components were present on the column from previous testing. The standard PPT run was to confirm retention time. Extracts were analyzed neat and with a PPT spike to confirm PPT presence by co-elution.

A chromatograph of the 50 μg/mL PPT standard analyzed by the initial HPLC screening method was developed. Extract samples were determined to be negative for PPT if chromatographs did not contain a peak overlapping the PPT standard peak. If samples contained a peak with a retention time similar to the PPT standard, they were further analyzed by the HPLC long method.

Sensitivity of this initial HPLC screening method for the detection of PPT was determined by analysis of PPT standard dilutions ranging from 0.001 mg/mL to 1 mg/mL prepared in DI water. The originally described gradient was changed to an isocratic run with a smaller injection volume because of the lower complexity and higher concentration of the PPT standard as compared to extract samples. A 10 min isocratic run was performed with an acetonitrile:DI water (50:50, v/v) mobile phase. The initial screening HPLC method was shown to have sensitivity for PPT down to a concentration of 2.5 μg/mL. A PPT concentration of 1 μg/mL was undetectable by this method.

Analysis of Soxhlet and SPE Extracts by HPLC Long Method

Due to the complexity of the samples and low concentrations, it was unclear from the initial screen if PPT was present in many of the samples. Therefore, after the initial screening described above, many samples were further analyzed by a longer, more complex HPLC method (Wong et al., 2000). The published method was modified to substitute spectral analysis for mass spectroscopy. This longer method was used to increase retention time and to obtain better separation of PPT from other sample components. Separation was carried out on an Alltech 4.6 mm i.d.×250 mm, 5 μm Hypersil® BDS column. Analysis of 20 μL of each extract was performed using a 55 min linear gradient from 65:35 H₂O:MeOH (HPLC grade) to 35:65 H₂O:MeOH. The flow rate was 1 mL/min. The separation was held at 35:65 H₂O: MeOH for 5 min at the end of each run. Spectrophotometric detection of PPT was at 240 nm using a diode array detector. A 0.05 mg/mL PPT standard in EtOH was analyzed each day and samples were run to confirm retention time and compare spectra. Extracts were analyzed neat and spectral data from the diode array detector was collected from 190-400 nm.

During development of the HPLC long method, it was discovered that PPT showed stronger absorbance at a wavelength below 254 nm. This wavelength had been obtained from the literature (Yoo and Porter, 1993) and was used for initial HPLC screening experiments. A wavelength of 240 nm was chosen for the long method to maximize the absorbance signal from PPT and minimize any negative effects from solvent absorbances.

To determine the presence of PPT in samples analyzed by the HPLC long method, peak retention times were compared to a 50 μg/mL PPT standard. Samples containing peaks with retention times from 31-39 min were compared spectrally to the peak apex spectrum of the PPT standard.

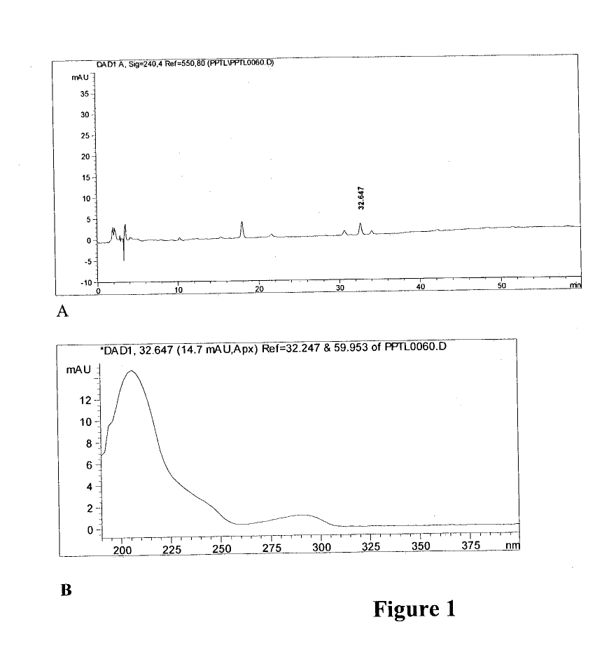

A wild rhizome SPE extract from the endophyte, PPAEE1 that was found to contain a peak at 32.6 min with a peak apex spectrum corresponding to that of the PPT standard is shown in FIG. 1.

Another wild rhizome SPE extract (from the endophyte, PPAEE2) was also found to contain a peak at 38.5 min (on a different HPLC instrument and column) with a peak apex spectrum corresponding to that of the PPT standard.

The SPE extracts from the endophytes, PPAEE1 and PPAEE2, have also shown the same UV absorption characteristics as authentic podophyllotoxin (data not shown).

For the HPLC long method, first, a wavelength of 240 nm was used as described above to detect PPT in extract samples. This wavelength was chosen to maximize the absorbance signal from PPT and minimize any negative effects from solvent absorbances. However, due to the strong absorbance signal of PPT at wavelengths below 240 nm and the presence of PPT in extremely low amounts, several extracts were rescreened at multiple wavelengths. Several extracts determined to be negative for the presence of PPT by the initial HPLC screening and HPLC long methods were reanalyzed by the HPLC long method. Analysis was performed using multiple detection wavelengths below 240 nm (John R. Porter and Rajeswari Dondapati, University of the Sciences in Philadelphia). This rescreening was done to ensure no extracts containing extremely low levels of PPT were overlooked by the previous analyses.

Confirmation of PPT by LC/MS

A sample found to be positive for the presence of PPT by HPLC and spectral analysis was independently confirmed by LC/MS (Lew Kilmer, GlaxoSmithKline). Analysis of the extract was carried out using an Agilent 1100 series HPLC with a diode array detector and a HP/Bruker ion trap mass spectrometer. Samples were separated on the previously described Alltech Hypersil® BDS column used for the long method analysis of extracts. A 25 μL sample was analyzed using a 40-min linear gradient from 5:95 Acetonitrile:H₂O (both containing 0.1% TFA) to 95:5 Acetonitrile:H₂O with a 1 mL/min flow rate. Detection was at 220 nm with a diode array detector. Peaks at 20.95-20.96 min were analyzed by the mass spectrometer using a scan range of 50-850 daltons and an accumulation time of 850 μs. Mass spectral data was collected and processed using Bruker Data Analysis software.

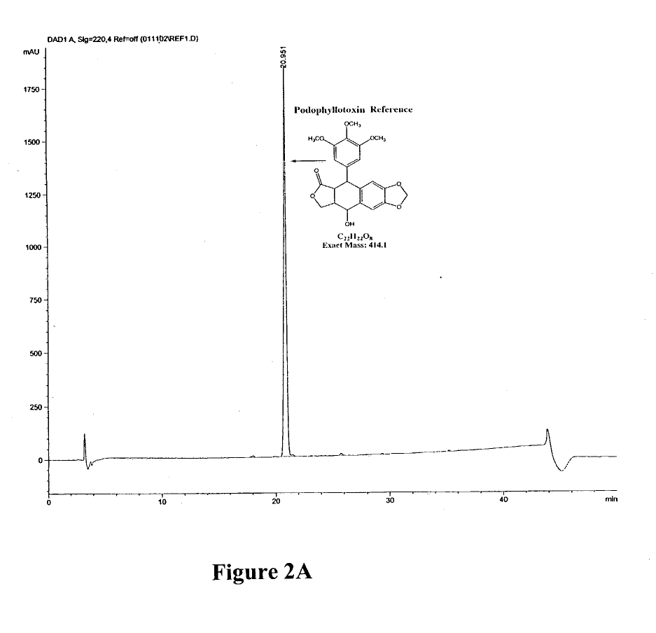

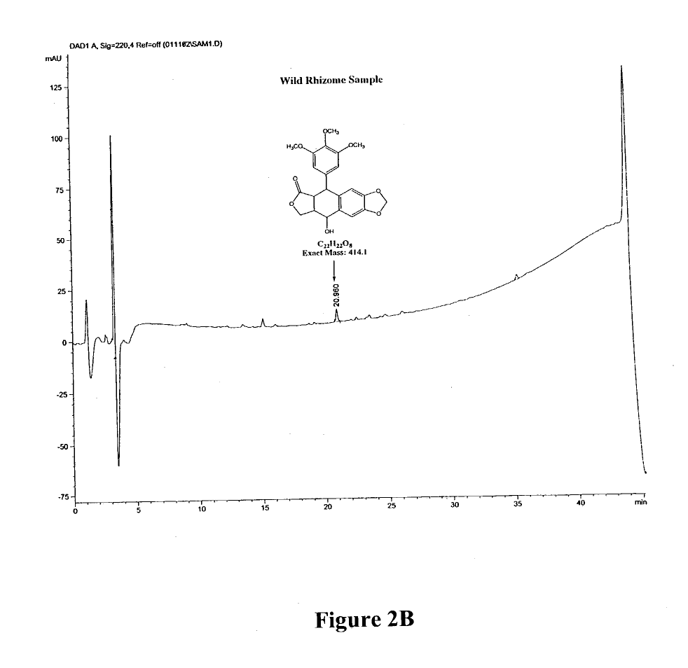

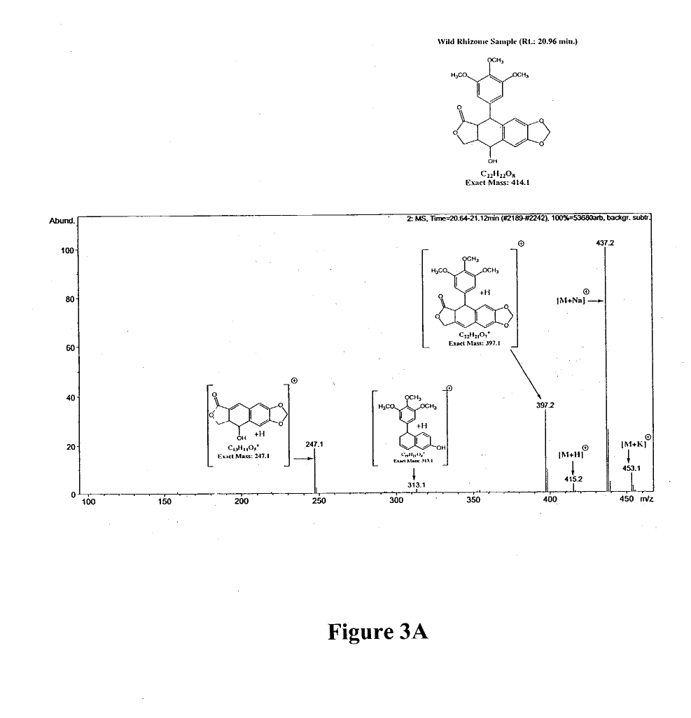

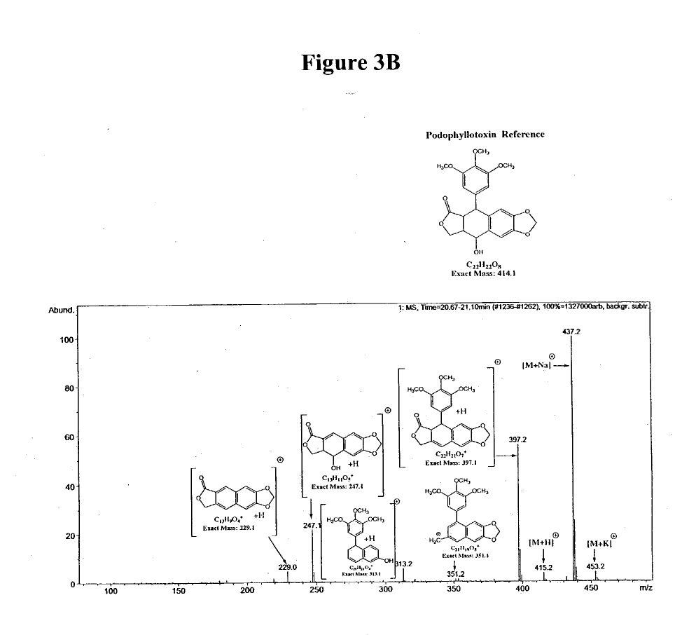

The presence of PPT in the Wild Rhizome SPE extract from the endophyte PPAEE1 was independently confirmed by LC/MS (Lew Kilmer, GlaxoSmithKline) as previously described in Materials and Methods. It was shown by LC/MS analysis that the SPE extract of Wild Rhizome matched that of the PPT standard. The LC/MS chromatographic analysis detected a peak with a retention time of 20.9 min in both the PPT standard (FIG. 2A) and the Wild Rhizome SPE extract (FIG. 2B). The mass spectrum of this peak at 20.9 in the Wild Rhizome SPE extract (FIG. 3A) showed the same pattern of fragment masses as the PPT standard mass spectrum (FIG. 3B).

The yield of the podophyllotoxin in the cultures is estimated to be 20 μg/L of medium.

### Example 2

### Brine Shrimp Lethality (BSL) Bioassay

In order to demonstrate the cytotoxicity of podophyllotoxin recovered from the endophytic fungi found in Podophyllum species, a simple brine shrimp lethality (BSL) bioassay was conducted to show the anti-tubulin cytotoxicity that would be expected for podophyllotoxin. BSL assay of the extracts from the endophytes PPAEE1 and PPAEE2 have shown cytotoxic activity which are consistent with the concentration of podophyllotoxin present in the extract (LD₅₀=3-6 μg/mL).

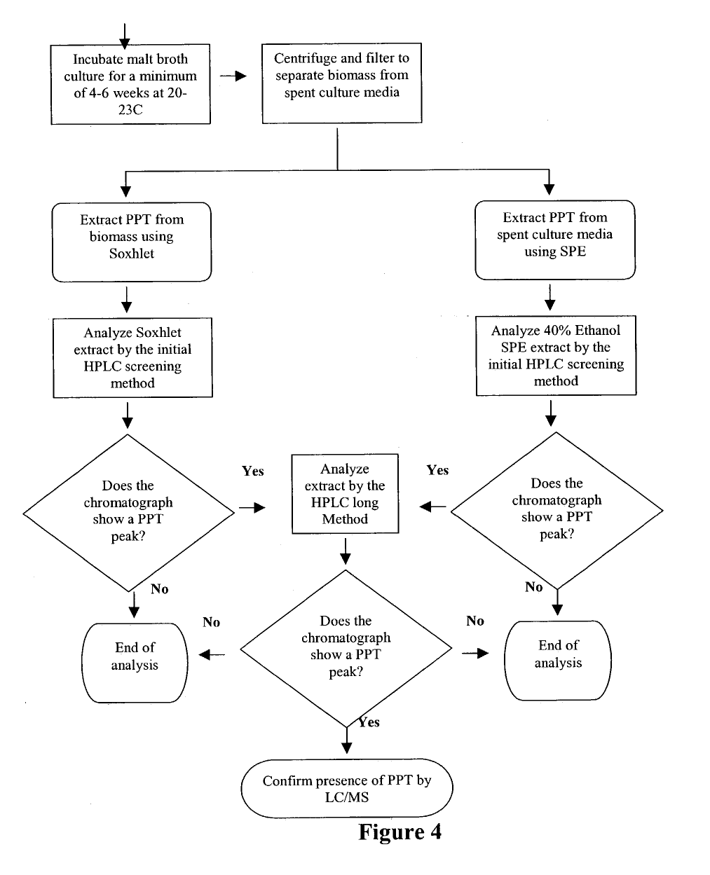

Shown in FIG. 4 is an exemplary scheme for the analysis of endophyte cultures for the presence of PPT.

### Example 3

### Deposit of Biological Materials

Viable samples of Phialocephala fortinii strains PPAEE1 and PPAEE2 isolated from Podophyllum peltatum have been deposited with the the American Type Culture Collection (ATCC), 10801 University Blvd., Manassas, Va. 20110-2209, as of May 27, 2003. The strain PPAEE1 has been assigned ATCC Accession No.PTA-5208 and the strain PPAEE2 has been assigned ATCC Accession No PTA-5209. The Phialocephala fortinii strains deposited are referred to herein as “the deposited strains.”

As a convenience to those skilled in the art, the deposit of the deposited strains has been made under the terms of the Budapest Treaty on the International Recognition of the Deposit of Micro-organisms for Purposes of Patent Procedure. All restrictions on the availability to the public of the deposited strains will be irrevocably removed upon the granting of a patent with the possible exception of requiring the request for the deposit be in the format specified in 37 CFR §1.808(b).

All publications and references, including but not limited to patents and patent applications, cited in this specification, are herein incorporated by reference in their entirety as if each individual publication or reference were specifically and individually indicated to be incorporated by reference herein as being fully set forth. While this invention has been described with a reference to specific embodiments, it will be obvious to those of ordinary skill in the art that variations in these methods and compositions may be used and that it is intended that the invention may be practiced otherwise than as specifically described herein. Accordingly, this invention includes all modifications encompassed within the spirit and scope of the invention as defined by the claims.

What is claimed is:

1. A biologically pure fungal strain capable of producing podophyllotoxin, wherein the fungal strain exists naturally as an endophytic fungus in a Podophyllum species.

2. The biologically pure fungal strain of claim 1, wherein the Podophyllum species is P. peltatum.

3. The biologically pure fungal strain of claim 2, wherein the strain is that designated as PPAEE1 which has been deposited at ATCC as Accession No. PTA-5208

4. The biologically pure fungal strain of claim 2, wherein the strain is that designated as PPAEE2 which has been deposited at ATCC as Accession No. PTA-5209.

5. A method for the production of podophyllotoxin comprising:
(a) culturing an endophyte fungus isolated from a Podophyllum species and capable of producing podophyllotoxin in a nutrient medium capable of supporting growth of the endophyte; and
(b) recovering the podophyllotoxin from the endophyte or medium in which the endophyte has grown.

6. The method of claim 5, wherein the culturing takes place in a fermentation culture.

7. The method of claim 5, wherein the Podophyllum species is P. peltatum.

8. The method of claim 7, wherein the endophyte fungus is that designated as PPAEE1 which has been deposited at ATCC as Accession No. PTA-5208.

9. The method of claim 7, wherein the endophyte fungus is that designated as PPAEE2 which has been deposited at ATCC as Accession No. PTA-5209.

10. The method of claim 5, wherein the fungus is cultured in the nutrient medium for a period of about 4 to 6 weeks.

11. A method for the production of podophyllotoxin which comprises:
cultivating a fungal strain, designated as PPAEE1 and which has been deposited at ATCC as Acession No. PTA-5208, in a culture medium comprising assimilable sources of carbon and nitrogen; and recovering the podophyllotoxin from the medium.

12. The method of claim 11, wherein the fungal strain is cultivated in a fermentation culture.

13. A method for the production of podophyllotoxin which comprises:
cultivating a fungal strain, designated as PPAEE2 and which has been deposited at ATCC as Accession No. PTA-5209, in a culture medium comprising assimilable sources of carbon and nitrogen; and recovering the podophyllotoxin from the medium.

14. The method of claim 13, wherein the fungal strain is cultivated in a fermentation culture.
